(19) United States
(12) Patent Application Publication
(10) Pub. No.: US 2025/0259733 A1
(43) Pub. Date: Aug. 14, 2025

(54) ANATOMICALLY AWARE VISION-LANGUAGE MODELS FOR MEDICAL IMAGING ANALYSIS

(71) Applicant: Siemens Healthineers AG, Forchheim (DE)

(72) Inventors: Alexandru Constantin Serban, Constanta (RO); Mehmet Akif Gulsun, Princeton, NJ (US); Vivek Singh, Princeton, NJ (US); Puneet Sharma, Princeton Junction, NJ (US)

(21) Appl. No.: 18/438,551

(22) Filed: Feb. 12, 2024

(51) Int. Cl. G16H 30/40; G06T 7/00; G06V 10/774

(52) U.S. Cl. CPC G16H 30/40; G06T 7/0012; G06V 10/774; G06T 2207/20081

(57) ABSTRACT

Systems and methods for performing one or more medical imaging analysis tasks using a vision-language model are provided. One or more input medical images are received. Image embeddings are extracted from the one or more input medical images. One or more medical imaging analysis tasks are performed based on the image embeddings extracted from the one or more input medical images using a trained vision-language model. Results of the one or more medical imaging analysis tasks are output. The trained vision-language model is trained by: receiving one or more training medical images and a text-based report associated with the one or more training medical images, extracting image embeddings from the one or more training medical images, generating one or more instructions based on the text-based report using a language model, and training the vision-language model to perform the one or more medical imaging analysis tasks based on the image embeddings extracted from the one or more training medical images and the one or more generated instructions.

### TECHNICAL FIELD

The present invention relates generally to machine learning-based medical imaging analysis, and in particular to anatomically aware VLMs (vision-language models) for medical imaging analysis.

### BACKGROUND

Recently, machine learning-based vision models have been proposed for performing various medical imaging analysis tasks. Conventionally, such vision models rely on pixel-based imaging data of medical images to directly extract insights for performing medical imaging analysis tasks. However, such conventional vision models inherently struggle to enforce even simple anatomical constraints (e.g., the precise positioning of the left atrium in the heart compared with the right atrium), let alone intricate anatomical constraints (e.g., the relative positioning of an organ to any other organ). Further, such conventional vision models struggle to enforce constraints for specific medical interpretations.

### BRIEF SUMMARY OF THE INVENTION

Embodiments described herein provide for a machine learning-based vision-language model that associates anatomical vision representations with semantic language representations for performing a medical imaging analysis task.

In accordance with one or more embodiments, systems and methods for performing one or more medical imaging analysis tasks using a vision-language model are provided. One or more input medical images are received. Image embeddings are extracted from the one or more input medical images. One or more medical imaging analysis tasks are performed based on the image embeddings extracted from the one or more input medical images using a trained vision-language model. Results of the one or more medical imaging analysis tasks are output. The trained vision-language model is trained by: receiving one or more training medical images and a text-based report associated with the one or more training medical images, extracting image embeddings from the one or more training medical images, generating one or more instructions based on the text-based report using a language model, and training the vision-language model to perform the one or more medical imaging analysis tasks based on the image embeddings extracted from the one or more training medical images and the one or more generated instructions.

In one embodiment, the one or more instructions are further generated based on a plurality of predefined templates. The plurality of predefined templates comprises different initial instructions for extracting information from the text-based report and generating the one or more instructions.

In one embodiment, the one or more instructions are generated for associating anatomical features depicted in the one or more training medical images with textual anatomical descriptors, for associating anatomical features depicted in the one or more training medical images with each other, and/or for associating textual anatomical descriptors with image findings of the one or more training medical images. The image findings may comprise quantitative image findings of the one or more input medical images.

In one embodiment, instruction embeddings representing the one or more instructions are generated. The image embeddings and the instruction embeddings are combined and the results of the one or more medical imaging analysis tasks are generated based on the combined image embeddings and instruction embeddings.

In accordance with one embodiment, systems and methods for training a vision-language model to perform one or more medical imaging analysis tasks are provided. One or more training medical images and a text-based report associated with the one or more training medical images are received. Image embeddings are extracted from the one or more training medical images. One or more instructions are generated based on the text-based report using a language model. A vision-language model is trained to perform one or more medical imaging analysis tasks based on the image embeddings extracted from the one or more training medical images and the one or more generated instructions.

In one embodiment, the one or more instructions are generated for associating anatomical features depicted in the one or more training medical images with textual anatomical descriptors, for associating anatomical features depicted in the one or more training medical images with each other, and/or for associating textual anatomical descriptors with image findings of the one or more training medical images. The image findings may comprise quantitative image findings of the one or more input medical images.

These and other advantages of the invention will be apparent to those of ordinary skill in the art by reference to the following detailed description and the accompanying drawings.

### BRIEF DESCRIPTION OF THE DRAWINGS

FIG. 1 shows a workflow for training a vision-language model for performing a medical imaging analysis task, in accordance with one or more embodiments;

FIG. 2 shows a method for training a vision-language model for performing a medical imaging analysis task, in accordance with one or more embodiments;

FIG. 3 shows a method for performing a medical imaging analysis task using a vision-language model, in accordance with one or more embodiments;

FIG. 4 shows an exemplary artificial neural network that may be used to implement one or more embodiments;

FIG. 5 shows a convolutional neural network that may be used to implement one or more embodiments;

FIG. 6 shows a schematic structure of a recurrent machine learning model that may be used to implement one or more embodiments; and

FIG. 7 shows a high-level block diagram of a computer that may be used to implement one or more embodiments.

### DETAILED DESCRIPTION

The present invention generally relates to methods and systems for anatomically aware vision-language models for medical imaging analysis. Embodiments of the present invention are described herein to give a visual understanding of such methods and systems. A digital image is often composed of digital representations of one or more objects (or shapes). The digital representation of an object is often described herein in terms of identifying and manipulating the objects. Such manipulations are virtual manipulations accomplished in the memory or other circuitry/hardware of a computer system. Accordingly, is to be understood that embodiments of the present invention may be performed within a computer system using data stored within the computer system. Further, reference herein to pixels of an image may refer equally to voxels of an image and vice versa.

Embodiments described herein provide for a universal anatomically aware vision-language model for medical imaging analysis and clinical decision support. Embodiments described herein utilize instruction tuning to refine the capacity of the vision-language model to understand visual signals and associate them with textual anatomical descriptors. Leveraging medical reports, embodiments described herein autonomously generate anatomically correct instructions for instructing the vision-language model for performing a medical imaging analysis task. By combining medical images with text-based reports through the use of a large language model, the vision-language model is trained for anatomical understanding, making the vision-language model less prone to errors. Advantageously, the vision-language model in accordance with embodiments described herein thereby perform medical imaging analysis tasks with increased accuracy and anatomical robustness as compared with conventional approaches.

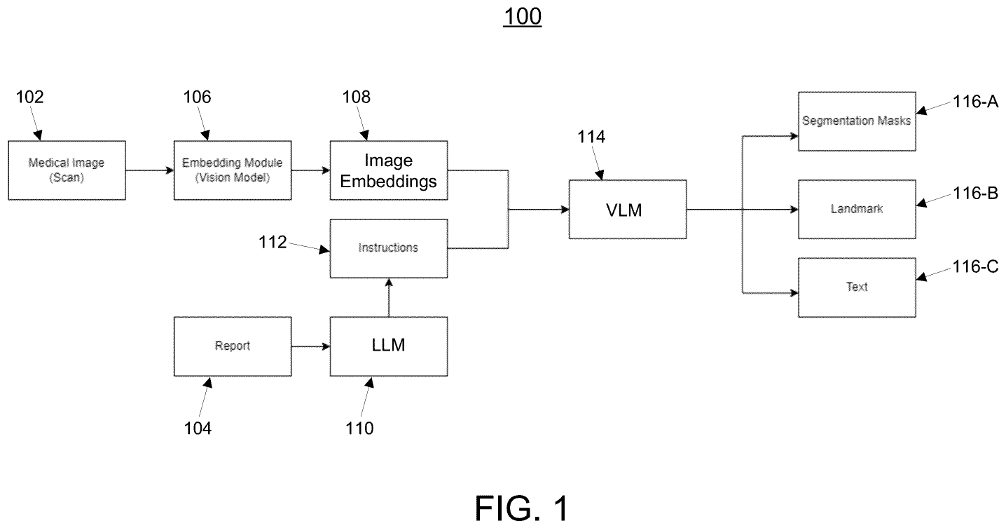

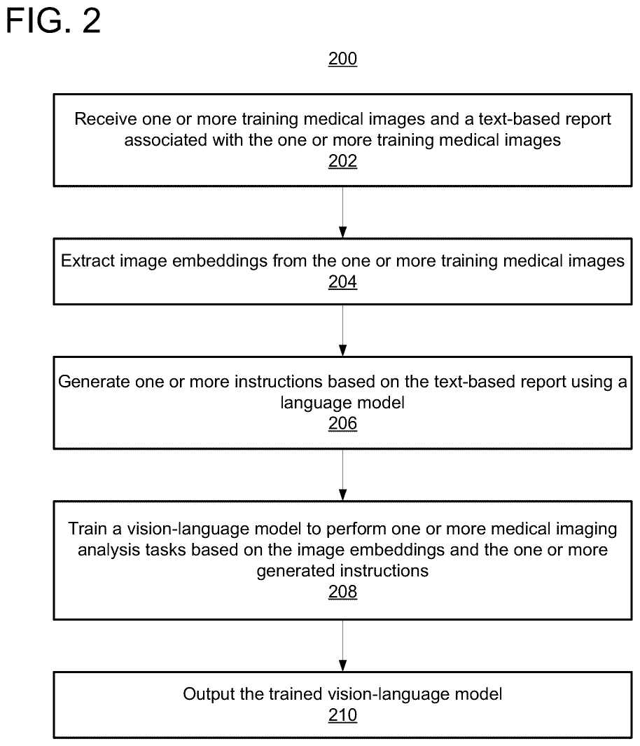

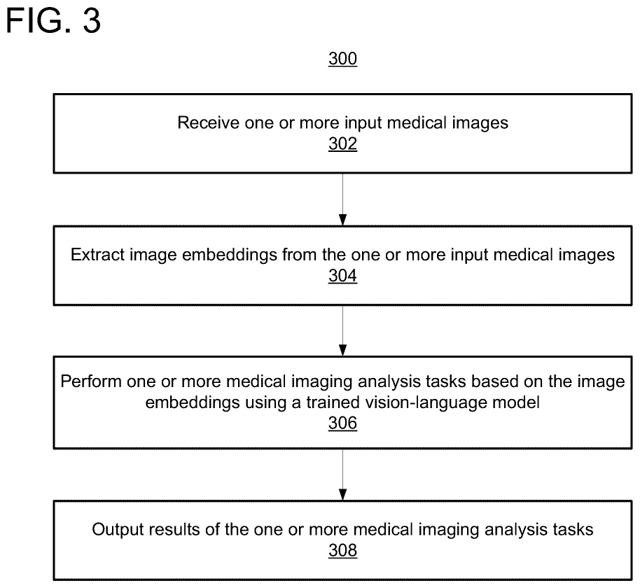

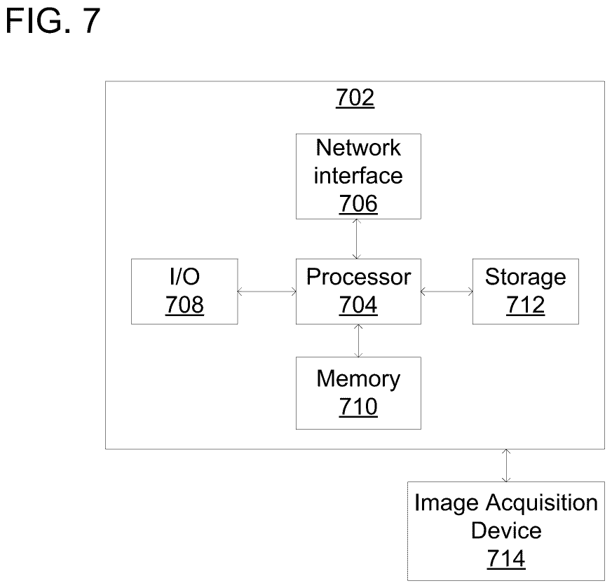

FIG. 1 shows a workflow 100 for training a vision-language model for performing a medical imaging analysis task, in accordance with one or more embodiments. FIG. 2 shows a method 200 for training a vision-language model for performing a medical imaging analysis task, in accordance with one or more embodiments. The steps of method 200 may be performed by one or more suitable computing devices, such as, e.g., computer 702 of FIG. 7. FIG. 1 and FIG. 2 will be described together. The steps/operations of workflow 100 of FIG. 1 and method 200 of FIG. 2 are performed during a prior offline or training stage for training the vision-language model. Once trained, the trained vision-language model is applied during an online or inference stage, e.g., to perform method 300 of FIG. 3.

At step 202 of FIG. 2, one or more training medical images and a text-based report associated with the one or more training medical images are received. In one example, as shown in workflow 100 of FIG. 1, the one or more training medical images is medical image 102 and the text-based report is report 104.

The one or more training medical images may depict one or more anatomical objects of interest, such as, e.g., organs, bones, vessels, tumors or abnormalities or pathologies, or any other suitable anatomical object or objects of interest of a patient. The one or more training medical images may be of any suitable imaging modality, such as, e.g., CT (computed tomography), MRI (magnetic resonance imaging), US (ultrasound), x-ray (e.g., angiography or fluoroscopy), PET (positron emission tomography), SPECT (single-photon emission computed tomography), or any other medical imaging modality or combinations of medical imaging modalities. The one or more training medical images may be 2D (two dimensional) images and/or 3D (three dimensional) volumes. The one or more training medical images may be annotated. For example, the annotations may be expert annotations, predictions generated by vision models, information extracted from text-based reports, longitudinal clinical data (e.g., disease progression or events), etc.

The text-based report comprises text associated with the one or more training medical images. The text-based report may comprise, for example, demographic information, vital signs, medical history, family history, laboratory results, medications, measurements and information extracted from medical images, etc. of the patient. In one embodiment, the text-based report may be a medical report comprising medical findings of the one or more training medical images. In one embodiment, the text-based report comprises textual anatomical descriptors of the anatomical objects depicted in the one or more training medical images. The text-based report may be manually generated by a user (e.g., a clinical) or automatically generated by, e.g., a machine learning based model.

The one or more training medical images and the text-based report may be received, for example, by directly receiving the one or more training medical images from an image acquisition device (e.g., image acquisition device 714 of FIG. 7) as the images are acquired, by loading the one or more training medical images and/or the text-based report from a storage or memory of a computer system (e.g., memory 710 or storage 712 of computer 702 of FIG. 7), or by receiving the one or more training medical images and/or the text-based report from a remote computer system (e.g., computer 702 of FIG. 7). Such a computer system or remote computer system may comprise one or more patient databases, such as, e.g., an EHR (electronic health record), EMR (electronic medical record), PHR (personal health record), HIS (health information system), RIS (radiology information system), PACS (picture archiving and communication system), LIMS (laboratory information management system), or any other suitable database or system.

At step 204 of FIG. 2, image embeddings are extracted from the one or more training medical images. In one embodiment, the image embeddings are extracted from the one or more training medical images using a machine learning-based feature extraction model. In one example, as shown in workflow 100 of FIG. 1, the image embeddings are image embeddings 108 extracted from medical image 102 using embedding module (vision model) 106. However, the image embeddings may be extracted from the one or more training medical images using any other suitable approach.

The machine learning-based feature extraction model may be a pre-trained vision model, such as, e.g., an autoencoder. The machine learning-based feature extraction network receives as input the one or more training medical images and generates as output the image embeddings. The image embeddings are low-level latent features representing the one or more training medical images as numerical feature vectors. The image embeddings operate analogously to a token in a text embedding process.

At step 206 of FIG. 2, one or more instructions are generated based on the text-based report using a language model. In one embodiment, the language model is an LLM (large language model). In one example, as shown in workflow 100 of FIG. 1, the one or more instructions are instructions 112 generated by LLM 110 based on report 104. However, the language model may be any other suitable machine learning-based language model (e.g., a small language model).

The one or more generated instructions are guidelines or directions provided to guide the behavior and output of a vision-language model. The one or more generated instructions provide anatomical clues to the vision-language model such that the vision-language model learns to associate the anatomical clues with information from the one or more training medical images. The one or more generated instructions may include, e.g., commands, questions, constraints, requirements, contextual information, or any other guideline or direction guiding the behavior and output of the vision-language model. In one example, the one or more generated instructions comprise: “segment the most calcified artery from the given image,” which involves understanding of arteries and calcifications and quantitative estimates for calcifications. In another example, the one or more generated instructions instruct the language model to segment a certain artery (e.g., “segment the circumflex branch of the left coronary artery in the given image”) or to detect disease (e.g., “is there a plaque on the left anterior descending artery main branch in the given image”), which involve understanding of anatomical segments and classification of plaques. In a further example, the one or more generated instructions may inquire about therapy planning: “will a 20 millimeter stent fully cover the proximal left anterior descending artery lesion?” which involves understanding of anatomical segments, lesion length, and quantitative comparison. The one or more generated instructions are generated as instruction embeddings. The instruction embeddings are low-level latent features representing the one or more generated instructions as numerical feature vectors.

In one embodiment, a templating technique is applied to generate the one or more instructions. In this embodiment, the language model is queried according to a plurality of predefined templates. The plurality of predefined templates comprises different initial instructions for extracting certain information from the text-based report and/or for generating the one or more instructions. In one embodiment, the plurality of predefined templates may comprise a template including a question and a plurality of possible answer choices. For example, the question may be “which of the following plurality of possible answer choices is the calcified part of an artery” and the plurality of possible answer choices may be (1) left diagonal, (2) left circumflex, (3) left main branch, and (4) distal circumflex. In another example, the plurality of predefined templates may help identify whether a pathology is present. The language model will identify from the text-based report which artery there is calcification and generate instructions saying “segment the artery with a calcification.” In a further example, given more context such as knowing the artery is a side branch of the right coronary artery, the plurality of predefined templates may comprise a template including the instructions “segment only the diagonal branch of the right coronary artery that has calcification.” Using this information, the vision-language model may be trained to know what a diagonal is, which is the right coronary artery, etc. The plurality of predefined templates may be generated by a user or may be generated according to any other suitable approach. The language model receives as input one or more prompts comprising the text-based report and the plurality of predefined templates and generates as output the instruction embeddings representing the one or more generated instructions. A prompt refers to input to the language model for generating a response. The prompt may be received from a computer system via one or more APIs (application programming interfaces) or from a user interacting with a computer system.

In one embodiment, the one or more generated instructions are organized as a hierarchical curriculum, where the instructions become progressively more complex. In one example, in the context of the coronary arteries, a first set of instructions may associate anatomical features depicted in the one or more input medical images with textual anatomical descriptors embedded within the first language model. For example, the first set of instructions may comprise: 1) “segment the left anterior descending main artery in the given image,” which would generate a segmentation mask of the left anterior descending main artery, 2) “segment the aortic root in the given image,” which would generate a segmentation mask of the aortic root,” etc. Like textual anatomical descriptors, associating different anatomical features depicted in the one or more input medical images with each other may also be relevant. Hence, a second set of instructions may associate anatomical features of the entire cardiac structure (e.g., the four chambers, the valves, the myocardium, etc.). Once the anatomical features are associated, a third set of instructions may focus on image findings to, e.g., associate textual anatomical descriptors with image findings (e.g., lesions, calcifications, occlusions, etc.) of the one or more input medical images. For example, the third set of instructions may comprise: “find the calcifications in the left anterior descending main artery in the given image,” which would generate a mask of the calcification. The anatomical features may comprise features relating to any anatomical object, such as, e.g., organs, bones, vessels, tumors or abnormalities or pathologies, or any other suitable anatomical object or objects of interest of a patient. In some embodiments, the one or more instructions may comprise binary instructions, such as: “is there a calcification present in left coronary artery,” which would generate a binary yes or no response.

In one embodiment, the image findings may comprise quantitative image findings such that the one or more instructions associate textual anatomical descriptors with the quantitative image findings (e.g., a size of a lesion, lesion volume, calcification volume score, etc.) of the one or more input medical images. In this embodiment, the first language model may be prompted to, e.g., determine one or more measurement over the segmentation masks and generate one or more instructions associated with the measurements. One example of such instructions is: “what is the size of the lesion on the right coronary artery branch in the given image?” which would generate the segmentation of the lesion and report the size of the lesion based on the segmentation.

Where the language model is an LLM, the LLM may be any suitable pre-trained deep learning based LLM. For example, the LLM may be based on the transformer architecture, which uses a self-attention mechanism to capture long-range dependencies in text. One example of a transformer-based architecture is GPT (generative pre-training transformer), which has a multilayer transformer decoder architecture that may be pretrained to optimize the next token prediction task and then fine-tuned with labelled data for various downstream tasks. GPT-based LLMs may be trained and/or fine-tuned using reinforcement learning with human feedback for performing various natural language processing tasks. Other exemplary transformer-based architectures include BLOOM (BigScience Large Open-science Open-access Multilingual Language Model) and BERT (Bidirectional Encoder Representations from Transformers). In some embodiments, the LLM may be a multi-modal LLM to receive, e.g., imaging data (e.g., the one or more input medical images) in addition to the text-based report and the plurality of predefined templates.

At step 208 of FIG. 2, a vision-language model is trained to perform one or more medical imaging analysis tasks based on the image embeddings and the one or more generated instructions. The one or more medical imaging analysis tasks may comprise any suitable medical imaging analysis task or tasks, such as, e.g., segmentation, classification, detection, quantification, etc. In one example, as shown in workflow 100 of FIG. 1, the vision-language model is VLM 114 for performing the medical imaging analysis tasks of segmentation, landmark detection, and report generation. VLM 114 generates segmentation masks 116-A, landmark 116-B, and text 116-C from image embeddings 108 and instructions 112.

The vision-language model is a multi-modal model that receives as input both text-based data and image-based data. The vision-language model may be any suitable pretrained deep learning based vision-language model. Examples of the vision-language model include CLIP (contrastive language-image pretraining), Flamingo, and VL-BERT (visual BERT).

To train the vision-language model to perform the medical imaging analysis task, the image embeddings and instruction embeddings are first combined (e.g., concatenated). The vision-language model receives as input one or more prompts comprising the combined image and instruction embeddings and generates as output results of the medical imaging analysis task. The vision-language model thus performs the medical imaging analysis task according to the one or more generated instructions represented by the instruction embeddings. The prompt may be received from a computer system via one or more APIs (application programming interfaces) or from a user interacting with a computer system. The vision-language model is trained based on the results of the medical imaging analysis tasks using any suitable loss function.

At step 210 of FIG. 2, the trained vision-language model is output. For example, the trained vision-language model can be output by storing the trained vision-language model on a memory or storage of a computer system (e.g., memory 710 or storage 712 of computer 702 of FIG. 7) or by transmitting the trained vision-language model to a remote computer system (e.g., computer 702 of FIG. 7).

FIG. 3 shows a method 300 for performing a medical imaging analysis task using a trained vision-language model, in accordance with one or more embodiments. The steps of method 300 may be performed by one or more suitable computing devices, such as, e.g., computer 702 of FIG. 7. The steps of method 300 of FIG. 3 are performed using the trained vision-language model during an online or inference stage. The trained vision-language model is trained during a prior offline or training stage, e.g., according to workflow 100 of FIG. 1 or method 200 of FIG. 2.

At step 302 of FIG. 3, one or more input medical images are received. The one or more input medical images may depict one or more anatomical objects of interest. The one or more input medical images may be of any suitable imaging modality, such as, e.g., CT, MRI, US, x-ray, PET, SPECT, etc., and may be 2D images and/or 3D volumes.

The one or more input medical images may be received, for example, by directly receiving the one or more input medical images from an image acquisition device (e.g., image acquisition device 714 of FIG. 7) as the images are acquired, by loading the one or more input medical images from a storage or memory of a computer system (e.g., memory 710 or storage 712 of computer 702 of FIG. 7), or by receiving the one or more input medical images from a remote computer system (e.g., computer 702 of FIG. 7). Such a computer system or remote computer system may comprise one or more patient databases.

At step 304 of FIG. 3, image embeddings are extracted from the one or more input medical images. In one embodiment, the image embeddings are extracted from the one or more input medical images using a machine learning-based feature extraction model. For example, the machine learning-based feature extraction model may be a pre-trained vision model, such as, e.g., an autoencoder. However, the image embeddings may be extracted from the one or more training medical images using any other suitable approach. The machine learning-based feature extraction network receives as input the one or more input medical images and generates as output the image embeddings. The image embeddings are low-level latent features representing the one or more input medical images as numerical feature vectors.

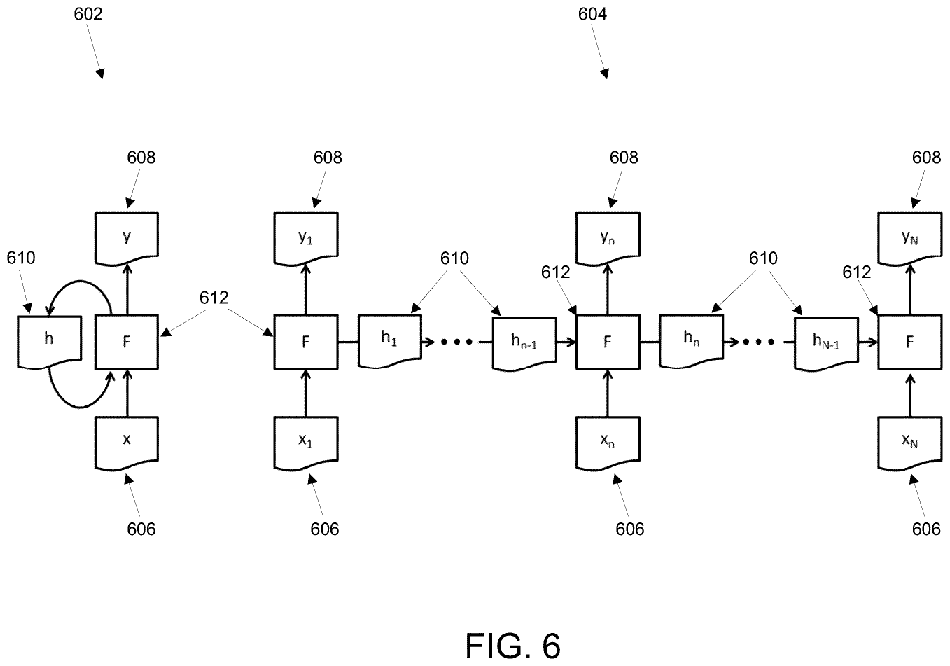

At step 306 of FIG. 6, one or more medical imaging analysis tasks are performed based on the image embeddings using a trained vision-language model. The medical imaging analysis task may comprise any suitable medical imaging analysis task or tasks, such as, e.g., segmentation, classification, detection, quantification, etc. The trained vision-language model receives as input the image embeddings and generates as output results of the one or more medical imaging analysis tasks. The vision-language model may be any suitable pretrained deep learning based vision-language model, such as, e.g., CLIP, Flamingo, and VL-BERT. In one embodiment, the trained vision-language model is trained according to workflow 100 of FIG. 1 or method 200 of FIG. 2.

At step 308 of FIG. 3, results of the one or more medical imaging analysis tasks are output. For example, the results of the one or more medical imaging analysis tasks can be output by displaying the results of the one or more medical imaging analysis tasks on a display device of a computer system (e.g., I/O 708 of computer 702 of FIG. 7), storing the results of the one or more medical imaging analysis tasks on a memory or storage of a computer system (e.g., memory 710 or storage 712 of computer 702 of FIG. 7), or by transmitting the results of the one or more medical imaging analysis tasks to a remote computer system (e.g., computer 702 of FIG. 7).

In one embodiment, where the trained vision-language model has been trained with extensive textual knowledge of, e.g., cardiovascular diseases, the trained vision-language model can be queried at step 306 of FIG. 3 in a broader context during inference. For example, the vision-language model may be queried with: “is the patient with the given cardiac exam image and Troponin-I 40 pg/mL value likely to have a MACE within the next 1 year?,” where pg/ml refers to picograms per milliliter and a MACE refers to a “major adverse cardiovascular event.”

Embodiments described herein are described with respect to the claimed systems as well as with respect to the claimed methods. Features, advantages or alternative embodiments herein can be assigned to the other claimed objects and vice versa. In other words, claims and embodiments for the systems can be improved with features described or claimed in the context of the respective methods. In this case, the functional features of the method are implemented by physical units of the system.

Furthermore, certain embodiments described herein are described with respect to methods and systems utilizing trained machine learning models, as well as with respect to methods and systems for providing trained machine learning models. Features, advantages or alternative embodiments herein can be assigned to the other claimed objects and vice versa. In other words, claims and embodiments for providing trained machine learning models can be improved with features described or claimed in the context of utilizing trained machine learning models, and vice versa. In particular, datasets used in the methods and systems for utilizing trained machine learning models can have the same properties and features as the corresponding datasets used in the methods and systems for providing trained machine learning models, and the trained machine learning models provided by the respective methods and systems can be used in the methods and systems for utilizing the trained machine learning models.

In general, a trained machine learning model mimics cognitive functions that humans associate with other human minds. In particular, by training based on training data the machine learning model is able to adapt to new circumstances and to detect and extrapolate patterns. Another term for “trained machine learning model” is “trained function.”

In general, parameters of a machine learning model can be adapted by means of training. In particular, supervised training, semi-supervised training, unsupervised training, reinforcement learning and/or active learning can be used. Furthermore, representation learning (an alternative term is “feature learning”) can be used. In particular, the parameters of the machine learning models can be adapted iteratively by several steps of training. In particular, within the training a certain cost function can be minimized. In particular, within the training of a neural network the backpropagation algorithm can be used.

In particular, machine learning models disclosed herein, such as, e.g., embedding module 106, LLM 110, and VLM 114 of FIG. 1, the feature extraction network utilized at step 204, the language model utilized at step 206, and the second language model utilized at step 208, and the vision-language model utilized at step 208 of FIG. 2, and the feature extraction network utilized at step 304 and the trained vision-language model utilized at step 306 of FIG. 3, can comprise, for example, a neural network. In particular, a neural network can be, e.g., a deep neural network, a convolutional neural network or a convolutional deep neural network. Furthermore, a neural network can be, e.g., an adversarial network, a deep adversarial network and/or a generative adversarial network.

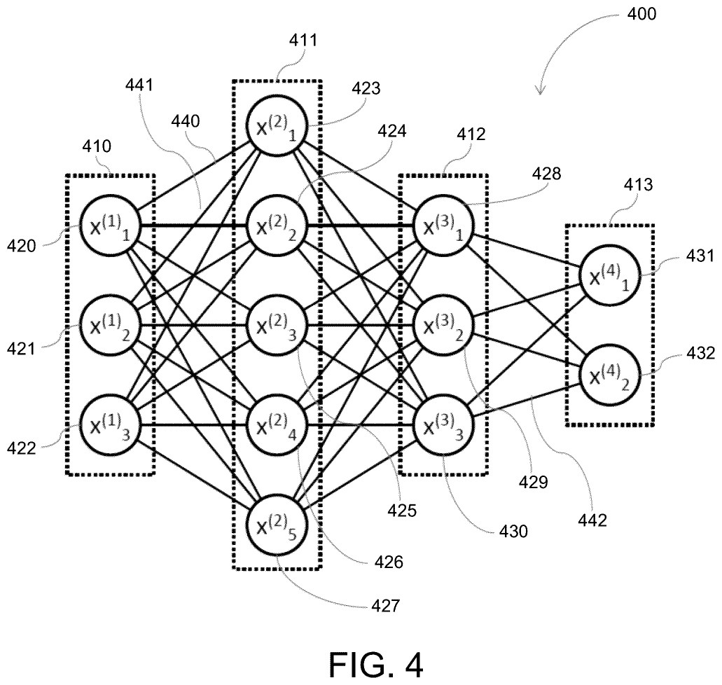

FIG. 4 shows an embodiment of an artificial neural network 400 that may be used to implement one or more machine learning models described herein. Alternative terms for “artificial neural network” are “neural network”, “artificial neural net” or “neural net”.

The artificial neural network 400 comprises nodes 420, . . . , 432 and edges 440, . . . 442, wherein each edge 440, . . . , 442 is a directed connection from a first node 420, . . . , 432 to a second node 420, . . . , 432. In general, the first node 420, . . . , 432 and the second node 420, . . . , 432 are different nodes 420, . . . , 432, it is also possible that the first node 420, . . . 432 and the second node 420, . . . , 432 are identical. For example, in FIG. 4 the edge 440 is a directed connection from the node 420 to the node 423, and the edge 442 is a directed connection from the node 430 to the node 432. An edge 440, . . . , 442 from a first node 420, 432 to a second node 420, . . . , 432 is also denoted as “ingoing edge” for the second node 420, . . . 432 and as “outgoing edge” for the first node 420, . . . , 432.

In this embodiment, the nodes 420, . . . , 432 of the artificial neural network 400 can be arranged in layers 410, . . . , 413, wherein the layers can comprise an intrinsic order introduced by the edges 440, . . . , 442 between the nodes 420, . . . , 432. In particular, edges 440, . . . , 442 can exist only between neighboring layers of nodes. In the displayed embodiment, there is an input layer 410 comprising only nodes 420, . . . , 422 without an incoming edge, an output layer 413 comprising only nodes 431, 432 without outgoing edges, and hidden layers 411, 412 in-between the input layer 410 and the output layer 413. In general, the number of hidden layers 411, 412 can be chosen arbitrarily. The number of nodes 420, . . . , 422 within the input layer 410 usually relates to the number of input values of the neural network, and the number of nodes 431, 432 within the output layer 413 usually relates to the number of output values of the neural network.

In particular, a (real) number can be assigned as a value to every node 420, . . . , 432 of the neural network 400. Here, x⁽ⁿ⁾^{ᵢ} denotes the value of the i-th node 420, . . . , 432 of the n-th layer 410, . . . , 413. The values of the nodes 420, . . . , 422 of the input layer 410 are equivalent to the input values of the neural network 400, the values of the nodes 431, 432 of the output layer 413 are equivalent to the output value of the neural network 400. Furthermore, each edge 440, . . . , 442 can comprise a weight being a real number, in particular, the weight is a real number within the interval [−1, 1] or within the interval [0, 1]. Here, w^{(m,n)}_{i,j} denotes the weight of the edge between the i-th node 420, . . . , 432 of the m-th layer 410, . . . , 413 and the j-th node 420, . . . , 432 of the n-th layer 410, . . . , 413. Furthermore, the abbreviation w⁽ⁿ⁾_{i,j} is defined for the weight w^{(n,n+1)}_{i,j}.

In particular, to calculate the output values of the neural network 400, the input values are propagated through the neural network. In particular, the values of the nodes 420, . . . , 432 of the (n+1)-th layer 410, . . . , 413 can be calculated based on the values of the nodes 420, . . . , 432 of the n-th layer 410, . . . , 413 by x⁽ⁿ⁺¹⁾ ^{ⱼ}=f(Σᵢx⁽ⁿ⁾^{ᵢ}·w⁽ⁿ⁾^{_{i,j}}).

Herein, the function f is a transfer function (another term is “activation function”). Known transfer functions are step functions, sigmoid function (e.g., the logistic function, the generalized logistic function, the hyperbolic tangent, the Arctangent function, the error function, the smoothstep function) or rectifier functions. The transfer function is mainly used for normalization purposes.

In particular, the values are propagated layer-wise through the neural network, wherein values of the input layer 410 are given by the input of the neural network 400, wherein values of the first hid-den layer 411 can be calculated based on the values of the input layer 410 of the neural network, wherein values of the second hidden layer 412 can be calculated based in the values of the first hidden layer 411, etc.

In order to set the values w^{(m,n)}^{_{i,j}} for the edges, the neural network 400 has to be trained using training data. In particular, training data comprises training input data and training output data (denoted as tᵢ). For a training step, the neural network 400 is applied to the training input data to generate calculated output data. In particular, the training data and the calculated output data comprise a number of values, said number being equal with the number of nodes of the output layer.

In particular, a comparison between the calculated output data and the training data is used to recursively adapt the weights within the neural network 400 (backpropagation algorithm). In particular, the weights are changed according to

$${w}^{{′ ( n )}_{i , j}} = {w}^{{( n )}_{i , j}} - γ \cdot {δ}^{{( n )}_{j}} \cdot {x}^{{( n )}_{i}}$$

wherein γ is a learning rate, and the numbers γ⁽ⁿ⁾ⱼ can be recursively calculated as

$${δ}^{{( n )}_{j}} = ( {\sum }_{k} {δ}^{{( n + 1 )}_{k}} \cdot {w}^{{( n + 1 )}_{j , k}} ) \cdot {f}^{′} ( {\sum }_{i} {x}^{{( n )}_{i}} \cdot {w}^{{( n )}_{i , j}} )$$

based on δ⁽ⁿ⁺¹⁾ⱼ, if the (n+1)-th layer is not the output layer, and

$${δ}^{{( n )}_{j}} = ( {x}^{{( n + 1 )}_{j}} - {t}^{{( n + 1 )}_{j}} ) \cdot {f}^{′} ( {x}^{{( n )}_{i}} \cdot {w}^{{( n )}_{i , j}} )$$

if the (n+1)-th layer is the output layer 413, wherein f′ is the first derivative of the activation function, and t⁽ⁿ⁺¹⁾ⱼ is the comparison training value for the j-th node of the output layer 413.

A convolutional neural network is a neural network that uses a convolution operation instead general matrix multiplication in at least one of its layers (so-called “convolutional layer”). In particular, a convolutional layer performs a dot product of one or more convolution kernels with the convolutional layer's input data/image, wherein the entries of the one or more convolution kernel are the parameters or weights that are adapted by training. In particular, one can use the Frobenius inner product and the ReLU activation function. A convolutional neural network can comprise additional layers, e.g., pooling layers, fully connected layers, and normalization layers.

By using convolutional neural networks input images can be processed in a very efficient way, because a convolution operation based on different kernels can extract various image features, so that by adapting the weights of the convolution kernel the relevant image features can be found during training. Furthermore, based on the weight-sharing in the convolutional kernels less parameters need to be trained, which prevents overfitting in the training phase and allows to have faster training or more layers in the network, improving the performance of the network.

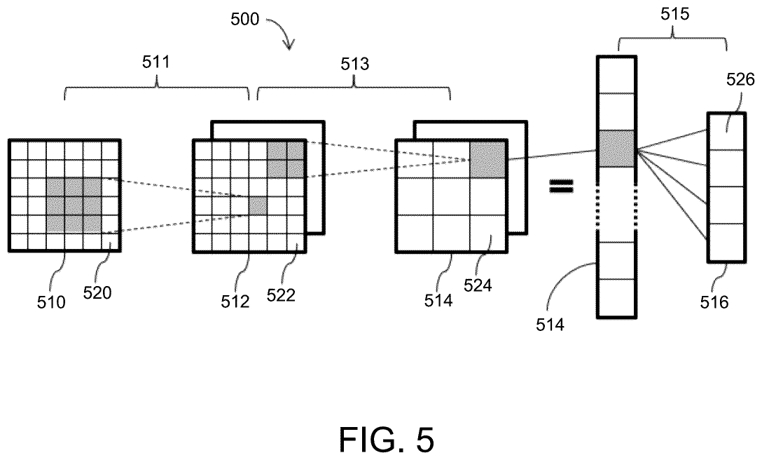

FIG. 5 shows an embodiment of a convolutional neural network 500 that may be used to implement one or more machine learning models described herein. In the displayed embodiment, the convolutional neural network comprises 500 an input node layer 510, a convolutional layer 511, a pooling layer 513, a fully connected layer 514 and an output node layer 516, as well as hidden node layers 512, 514. Alternatively, the convolutional neural network 500 can comprise several convolutional layers 511, several pooling layers 513 and several fully connected layers 515, as well as other types of layers. The order of the layers can be chosen arbitrarily, usually fully connected layers 515 are used as the last layers before the output layer 516.

In particular, within a convolutional neural network 500 nodes 520, 522, 524 of a node layer 510, 512, 514 can be considered to be arranged as a d-dimensional matrix or as a d-dimensional image. In particular, in the two-dimensional case the value of the node 520, 522, 524 indexed with i and j in the n-th node layer 510, 512, 514 can be denoted as x(n) [i, j]. However, the arrangement of the nodes 520, 522, 524 of one node layer 510, 512, 514 does not have an effect on the calculations executed within the convolutional neural network 500 as such, since these are given solely by the structure and the weights of the edges.

A convolutional layer 511 is a connection layer between an anterior node layer 510 (with node values x(n−1)) and a posterior node layer 512 (with node values x(n)). In particular, a convolutional layer 511 is characterized by the structure and the weights of the incoming edges forming a convolution operation based on a certain number of kernels. In particular, the structure and the weights of the edges of the convolutional layer 511 are chosen such that the values x(n) of the nodes 522 of the posterior node layer 512 are calculated as a convolution x(n)=K*x(n−1) based on the values x(n−1) of the nodes 520 anterior node layer 510, where the convolution * is defined in the two-dimensional case as

$${x}_{k}^{( n )} [ i , j ] = ( \begin{matrix}K & {}^{*} {x}^{( n - 1 )}\end{matrix} ) [ i , j ] = {\sum }_{{i}^{′}} {\sum }_{{j}^{′}} K [ {i}^{′} , {j}^{′} ] \cdot {x}^{( n - 1 )} [ i - {i}^{′} , j - {j}^{′} ] .$$

Here the kernel K is a d-dimensional matrix (in this embodiment, a two-dimensional matrix), which is usually small compared to the number of nodes 520, 522 (e.g., a 3×3 matrix, or a 5×5 matrix). In particular, this implies that the weights of the edges in the convolution layer 511 are not independent, but chosen such that they produce said convolution equation. In particular, for a kernel being a 3×3 matrix, there are only 9 independent weights (each entry of the kernel matrix corresponding to one independent weight), irrespectively of the number of nodes 520, 522 in the anterior node layer 510 and the posterior node layer 512.

In general, convolutional neural networks 500 use node layers 510, 512, 514 with a plurality of channels, in particular, due to the use of a plurality of kernels in convolutional layers 511. In those cases, the node layers can be considered as (d+1)-dimensional matrices (the first dimension indexing the channels). The action of a convolutional layer 511 is then a two-dimensional example defined as

$${x}^{{( n )}_{b}} [ i , j ] = {\sum }_{a} {K}_{a b} {}^{*} {x}^{{( n - 1 )}_{a}} [ i , j ] = {\sum }_{a} {\sum }_{{i}^{′}} {\sum }_{{j}^{′}} {K}_{a , b} [ {i}^{′} , {j}^{′} ] \cdot {x}^{{( n - 1 )}_{a}} [ i - {i}^{′} , j - {j}^{′} ]$$

where x⁽ⁿ⁻¹⁾^{ₐ} corresponds to the a-th channel of the anterior node layer 510, x⁽ⁿ⁾^{_{b}} corresponds to the b-th channel of the posterior node layer 512 and K_{a,b} corresponds to one of the kernels. If a convolutional layer 511 acts on an anterior node layer 510 with A channels and outputs a posterior node layer 512 with B channels, there are A·B independent d-dimensional kernels K_{a,b}.

In general, in convolutional neural networks 500 activation functions are used. In this embodiment re ReLU (acronym for “Rectified Linear Units”) is used, with R(z)=max(0, z), so that the action of the convolutional layer 511 in the two-dimensional example is

$${x}^{{( n )}_{b}} [ i , j ] = R ( {\sum }_{a} ( {K}_{a , b} * {x}^{{( n - 1 )}_{a}} ) [ i , j ] ) = R ( {\sum }_{a} {\sum }_{{i}^{′}} {\sum }_{{j}^{′}} {K}_{a , b} [ {i}^{′} , {j}^{′} ] \cdot {x}^{{( n - 1 )}_{a}} [ i - {i}^{′} , j - {j}^{′} ] )$$

It is also possible to use other activation functions, e.g., ELU (acronym for “Exponential Linear Unit”), LeakyReLU, Sigmoid, Tanh or Softmax.

In the displayed embodiment, the input layer 510 comprises 36 nodes 520, arranged as a two-dimensional 6×6 matrix. The first hidden node layer 512 comprises 72 nodes 522, arranged as two two-dimensional 6×6 matrices, each of the two matrices being the result of a convolution of the values of the input layer with a 3×3 kernel within the convolutional layer 511. Equivalently, the nodes 522 of the first hidden node layer 512 can be interpreted as arranged as a three-dimensional 2×6×6 matrix, wherein the first dimension correspond to the channel dimension.

The advantage of using convolutional layers 511 is that spatially local correlation of the input data can exploited by enforcing a local connectivity pattern between nodes of adjacent layers, in particular by each node being connected to only a small region of the nodes of the preceding layer.

A pooling layer 513 is a connection layer between an anterior node layer 512 (with node values x(n−1)) and a posterior node layer 514 (with node values x(n)). In particular, a pooling layer 513 can be characterized by the structure and the weights of the edges and the activation function forming a pooling operation based on a non-linear pooling function f. For example, in the two-dimensional case the values x(n) of the nodes 524 of the posterior node layer 514 can be calculated based on the values x(n−1) of the nodes 522 of the anterior node layer 512 as

$${x}^{{( n )}_{b}} [ i , j ] = f ( {x}^{( n - 1 )} [ {id}_{1} , {jd}_{2} ] , … , {x}^{{( n - 1 )}_{b}} [ ( i + 1 ) {d}_{1} - 1 , ( j + 1 ) {d}_{2} - 1 ] )$$

In other words, by using a pooling layer 513 the number of nodes 522, 524 can be reduced, by re-placing a number d1·d2 of neighboring nodes 522 in the anterior node layer 512 with a single node 522 in the posterior node layer 514 being calculated as a function of the values of said number of neighboring nodes. In particular, the pooling function f can be the max-function, the average or the L2-Norm. In particular, for a pooling layer 513 the weights of the incoming edges are fixed and are not modified by training.

The advantage of using a pooling layer 513 is that the number of nodes 522, 524 and the number of parameters is reduced. This leads to the amount of computation in the network being reduced and to a control of overfitting.

In the displayed embodiment, the pooling layer 513 is a max-pooling layer, replacing four neighboring nodes with only one node, the value being the maximum of the values of the four neighboring nodes. The max-pooling is applied to each d-dimensional matrix of the previous layer; in this embodiment, the max-pooling is applied to each of the two two-dimensional matrices, reducing the number of nodes from 72 to 18.

In general, the last layers of a convolutional neural network 500 are fully connected layers 515. A fully connected layer 515 is a connection layer between an anterior node layer 514 and a posterior node layer 516. A fully connected layer 513 can be characterized by the fact that a majority, in particular, all edges between nodes 514 of the anterior node layer 514 and the nodes 516 of the posterior node layer are present, and wherein the weight of each of these edges can be adjusted individually.

In this embodiment, the nodes 524 of the anterior node layer 514 of the fully connected layer 515 are displayed both as two-dimensional matrices, and additionally as non-related nodes (indicated as a line of nodes, wherein the number of nodes was reduced for a better presentability). This operation is also denoted as “flattening”. In this embodiment, the number of nodes 526 in the posterior node layer 516 of the fully connected layer 515 smaller than the number of nodes 524 in the anterior node layer 514. Alternatively, the number of nodes 526 can be equal or larger.

Furthermore, in this embodiment the Softmax activation function is used within the fully connected layer 515. By applying the Softmax function, the sum the values of all nodes 526 of the output layer 516 is 1, and all values of all nodes 526 of the output layer 516 are real numbers between 0 and 1. In particular, if using the convolutional neural network 500 for categorizing input data, the values of the output layer 516 can be interpreted as the probability of the input data falling into one of the different categories.

In particular, convolutional neural networks 500 can be trained based on the backpropagation algorithm. For preventing overfitting, methods of regularization can be used, e.g., dropout of nodes 520, . . . , 524, stochastic pooling, use of artificial data, weight decay based on the L1 or the L2 norm, or max norm constraints.

According to an aspect, the machine learning model may comprise one or more residual networks (ResNet). In particular, a ResNet is an artificial neural network comprising at least one jump or skip connection used to jump over at least one layer of the artificial neural network. In particular, a ResNet may be a convolutional neural network comprising one or more skip connections respectively skipping one or more convolutional layers. According to some examples, the ResNets may be represented as m-layer ResNets, where m is the number of layers in the corresponding architecture and, according to some examples, may take values of 34, 50, 101, or 152. According to some examples, such an m-layer ResNet may respectively comprise (m−2)/2 skip connections.

A skip connection may be seen as a bypass which directly feeds the output of one preceding layer over one or more bypassed layers to a layer succeeding the one or more bypassed layers. Instead of having to directly fit a desired mapping, the bypassed layers would then have to fit a residual mapping “balancing” the directly fed output.

Fitting the residual mapping is computationally easier to optimize than the directed mapping. What is more, this alleviates the problem of vanishing/exploding gradients during optimization upon training the machine learning models: if a bypassed layer runs into such problems, its contribution may be skipped by regularization of the directly fed output. Using ResNets thus brings about the advantage that much deeper networks may be trained.

In particular, a recurrent machine learning model is a machine learning model whose output does not only depend on the input value and the parameters of the machine learning model adapted by the training process, but also on a hidden state vector, wherein the hidden state vector is based on previous inputs used on for the recurrent machine learning model. In particular, the recurrent machine learning model can comprise additional storage states or additional structures that incorporate time delays or comprise feedback loops.

In particular, the underlying structure of a recurrent machine learning model can be a neural network, which can be denoted as recurrent neural network. Such a recurrent neural network can be described as an artificial neural network where connections between nodes form a directed graph along a temporal sequence. In particular, a recurrent neural network can be interpreted as directed acyclic graph. In particular, the recurrent neural network can be a finite impulse recurrent neural network or an infinite impulse recurrent neural network (wherein a finite impulse network can be unrolled and replaced with a strictly feedforward neural network, and an infinite impulse network cannot be unrolled and replaced with a strictly feedforward neural network).

In particular, training a recurrent neural network can be based on the BPTT algorithm (acronym for “backpropagation through time”), on the RTRL algorithm (acronym for “real-time recurrent learning”) and/or on genetic algorithms.

By using a recurrent machine learning model input data comprising sequences of variable length can be used. In particular, this implies that the method cannot be used only for a fixed number of input datasets (and needs to be trained differently for every other number of input datasets used as input), but can be used for an arbitrary number of input datasets. This implies that the whole set of training data, independent of the number of input datasets contained in different sequences, can be used within the training, and that training data is not reduced to training data corresponding to a certain number of successive input datasets.

FIG. 6 shows the schematic structure of a recurrent machine learning model F, both in a recurrent representation 602 and in an unfolded representation 604, that may be used to implement one or more machine learning models described herein. The recurrent machine learning model takes as input several input datasets x, x₁, . . . , x_{N} 606 and creates a corresponding set of output datasets y, y₁, . . . , y_{N} 608. Furthermore, the output depends on a so-called hidden vector h, h₁, . . . , h_{N} 610, which implicitly comprises information about input datasets previously used as input for the recurrent machine learning model F 612. By using these hidden vectors h, h₁, . . . , h_{N} 610, a sequentiality of the input datasets can be leveraged.

In a single step of the processing, the recurrent machine learning model F 612 takes as input the hidden vector hₙ₋₁ created within the previous step and an input dataset xₙ. Within this step, the recurrent machine learning model F generates as output an updated hidden vector hₙ and an output dataset yₙ. In other words, one step of processing calculates (yₙ, hₙ)=F(xₙ, hₙ₋₁), or by splitting the recurrent machine learning model F 612 into a part F(y) calculating the output data and F(h) calculating the hidden vector, one step of processing calculates yₙ=F^{(y)}(xₙ, hₙ₋₁) and hₙ=F^{(h)}(xₙ, hₙ₋₁). For the first processing step, h₀ can be chosen randomly or filled with all entries being zero. The parameters of the recurrent machine learning model F 612 that were trained based on training datasets before do not change between the different processing steps.

In particular, the output data and the hidden vector of a processing step depend on all the previous input datasets used in the previous steps. yₙ=F^{(y)}(xₙ, F^{(h)}(xₙ₋₁, hₙ₋₂)) and hₙ=F(h)(xₙ, F^{(h)}(xₙ₋₁, hₙ₋₂)).

Systems, apparatuses, and methods described herein may be implemented using digital circuitry, or using one or more computers using well-known computer processors, memory units, storage devices, computer software, and other components. Typically, a computer includes a processor for executing instructions and one or more memories for storing instructions and data. A computer may also include, or be coupled to, one or more mass storage devices, such as one or more magnetic disks, internal hard disks and removable disks, magneto-optical disks, optical disks, etc.

Systems, apparatuses, and methods described herein may be implemented using computers operating in a client-server relationship. Typically, in such a system, the client computers are located remotely from the server computer and interact via a network. The client-server relationship may be defined and controlled by computer programs running on the respective client and server computers.

Systems, apparatuses, and methods described herein may be implemented within a network-based cloud computing system. In such a network-based cloud computing system, a server or another processor that is connected to a network communicates with one or more client computers via a network. A client computer may communicate with the server via a network browser application residing and operating on the client computer, for example. A client computer may store data on the server and access the data via the network. A client computer may transmit requests for data, or requests for online services, to the server via the network. The server may perform requested services and provide data to the client computer(s). The server may also transmit data adapted to cause a client computer to perform a specified function, e.g., to perform a calculation, to display specified data on a screen, etc. For example, the server may transmit a request adapted to cause a client computer to perform one or more of the steps or functions of the methods and workflows described herein, including one or more of the steps or functions of FIGS. 1-3. Certain steps or functions of the methods and workflows described herein, including one or more of the steps or functions of FIGS. 1-3, may be performed by a server or by another processor in a network-based cloud-computing system. Certain steps or functions of the methods and workflows described herein, including one or more of the steps of FIGS. 1-3, may be performed by a client computer in a network-based cloud computing system. The steps or functions of the methods and workflows described herein, including one or more of the steps of FIGS. 1-3, may be performed by a server and/or by a client computer in a network-based cloud computing system, in any combination.

Systems, apparatuses, and methods described herein may be implemented using a computer program product tangibly embodied in an information carrier, e.g., in a non-transitory machine-readable storage device, for execution by a programmable processor; and the method and workflow steps described herein, including one or more of the steps or functions of FIGS. 1-3, may be implemented using one or more computer programs that are executable by such a processor. A computer program is a set of computer program instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

A high-level block diagram of an example computer 702 that may be used to implement systems, apparatuses, and methods described herein is depicted in FIG. 7. Computer 702 includes a processor 704 operatively coupled to a data storage device 712 and a memory 710. Processor 704 controls the overall operation of computer 702 by executing computer program instructions that define such operations. The computer program instructions may be stored in data storage device 712, or other computer readable medium, and loaded into memory 710 when execution of the computer program instructions is desired. Thus, the method and workflow steps or functions of FIGS. 1-3 can be defined by the computer program instructions stored in memory 710 and/or data storage device 712 and controlled by processor 704 executing the computer program instructions. For example, the computer program instructions can be implemented as computer executable code programmed by one skilled in the art to perform the method and workflow steps or functions of FIGS. 1-3. Accordingly, by executing the computer program instructions, the processor 704 executes the method and workflow steps or functions of FIGS. 1-3. Computer 702 may also include one or more network interfaces 706 for communicating with other devices via a network. Computer 702 may also include one or more input/output devices 708 that enable user interaction with computer 702 (e.g., display, keyboard, mouse, speakers, buttons, etc.).

Processor 704 may include both general and special purpose microprocessors, and may be the sole processor or one of multiple processors of computer 702. Processor 704 may include one or more central processing units (CPUs), for example. Processor 704, data storage device 712, and/or memory 710 may include, be supplemented by, or incorporated in, one or more application-specific integrated circuits (ASICs) and/or one or more field programmable gate arrays (FPGAs).

Data storage device 712 and memory 710 each include a tangible non-transitory computer readable storage medium. Data storage device 712, and memory 710, may each include high-speed random access memory, such as dynamic random access memory (DRAM), static random access memory (SRAM), double data rate synchronous dynamic random access memory (DDR RAM), or other random access solid state memory devices, and may include non-volatile memory, such as one or more magnetic disk storage devices such as internal hard disks and removable disks, magneto-optical disk storage devices, optical disk storage devices, flash memory devices, semiconductor memory devices, such as erasable programmable read-only memory (EPROM), electrically erasable programmable read-only memory (EEPROM), compact disc read-only memory (CD-ROM), digital versatile disc read-only memory (DVD-ROM) disks, or other non-volatile solid state storage devices.

Input/output devices 708 may include peripherals, such as a printer, scanner, display screen, etc. For example, input/output devices 708 may include a display device such as a cathode ray tube (CRT) or liquid crystal display (LCD) monitor for displaying information to the user, a keyboard, and a pointing device such as a mouse or a trackball by which the user can provide input to computer 702.

An image acquisition device 714 can be connected to the computer 702 to input image data (e.g., medical images) to the computer 702. It is possible to implement the image acquisition device 714 and the computer 702 as one device. It is also possible that the image acquisition device 714 and the computer 702 communicate wirelessly through a network. In a possible embodiment, the computer 702 can be located remotely with respect to the image acquisition device 714.

Any or all of the systems, apparatuses, and methods discussed herein may be implemented using one or more computers such as computer 702.

One skilled in the art will recognize that an implementation of an actual computer or computer system may have other structures and may contain other components as well, and that FIG. 7 is a high level representation of some of the components of such a computer for illustrative purposes.

Independent of the grammatical term usage, individuals with male, female or other gender identities are included within the term.

The foregoing Detailed Description is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the principles of the present invention and that various modifications may be implemented by those skilled in the art without departing from the scope and spirit of the invention. Those skilled in the art could implement various other feature combinations without departing from the scope and spirit of the invention.

The following is a list of non-limiting illustrative embodiments disclosed herein:

Illustrative embodiment 1. A computer-implemented method comprising: receiving one or more input medical images; extracting image embeddings from the one or more input medical images; performing one or more medical imaging analysis tasks based on the image embeddings extracted from the one or more input medical images using a trained vision-language model; and outputting results of the one or more medical imaging analysis tasks, wherein the trained vision-language model is trained by: receiving one or more training medical images and a text-based report associated with the one or more training medical images, extracting image embeddings from the one or more training medical images, generating one or more instructions based on the text-based report using a language model, and training the vision-language model to perform the one or more medical imaging analysis tasks based on the image embeddings extracted from the one or more training medical images and the one or more generated instructions.

Illustrative embodiment 2. The computer-implemented method of illustrative embodiment 1, wherein generating one or more instructions based on the text-based report using a language model comprises: generating the one or more instructions further based on a plurality of predefined templates, the plurality of predefined templates comprising different initial instructions for extracting information from the text-based report and generating the one or more instructions.

Illustrative embodiment 3. The computer-implemented method of any one of illustrative embodiments 1-2, wherein generating one or more instructions based on the text-based report using a language model comprises: generating the one or more instructions for associating anatomical features depicted in the one or more training medical images with textual anatomical descriptors.

Illustrative embodiment 4. The computer-implemented method of any one of illustrative embodiments 1-3, wherein generating one or more instructions based on the text-based report using a language model comprises: generating the one or more instructions for associating anatomical features depicted in the one or more training medical images with each other.

Illustrative embodiment 5. The computer-implemented method of any one of illustrative embodiments 1-4, wherein generating one or more instructions based on the text-based report using a language model comprises: generating the one or more instructions for associating textual anatomical descriptors with image findings of the one or more training medical images.

Illustrative embodiment 6. The computer-implemented method of illustrative embodiment 5, wherein the image findings comprise quantitative image findings of the one or more input medical images.

Illustrative embodiment 7. The computer-implemented method of any one of illustrative embodiments 1-6, wherein: generating one or more instructions based on the text-based report using a first language model comprises: generating instruction embeddings representing the one or more instructions; and training the vision-language model to perform the one or more medical imaging analysis tasks based on the image embeddings extracted from the one or more training medical images and the one or more generated instructions comprises: combining the image embeddings extracted from the one or more training medical images and the instruction embeddings, and generating results of the one or more medical imaging analysis tasks based on the combined image embeddings and instruction embeddings.

Illustrative embodiment 8. An apparatus comprising: means for receiving one or more input medical images; means for extracting image embeddings from the one or more input medical images; means for performing one or more medical imaging analysis tasks based on the image embeddings extracted from the one or more input medical images using a trained vision-language model; and means for outputting results of the one or more medical imaging analysis tasks, wherein the trained vision-language model is trained by: receiving one or more training medical images and a text-based report associated with the one or more training medical images, extracting image embeddings from the one or more training medical images, generating one or more instructions based on the text-based report using a language model, and training the vision-language model to perform the one or more medical imaging analysis tasks based on the image embeddings extracted from the one or more training medical images and the one or more generated instructions.

Illustrative embodiment 9. The apparatus of illustrative embodiment 8, wherein generating one or more instructions based on the text-based report using a language model comprises: generating the one or more instructions further based on a plurality of predefined templates, the plurality of predefined templates comprising different initial instructions for extracting information from the text-based report and generating the one or more instructions.

Illustrative embodiment 10. The apparatus of any one of illustrative embodiments 8-9, wherein generating one or more instructions based on the text-based report using a language model comprises: generating the one or more instructions for associating anatomical features depicted in the one or more training medical images with textual anatomical descriptors.

Illustrative embodiment 11. The apparatus of any one of illustrative embodiments 8-10, wherein the means for generating one or more instructions based on the text-based report using a language model comprises: generating the one or more instructions for associating anatomical features depicted in the one or more training medical images with each other.

Illustrative embodiment 12. A non-transitory computer-readable storage medium comprising instructions which, when executed by a computer, cause the computer to carry out operations comprising: receiving one or more input medical images; extracting image embeddings from the one or more input medical images; performing one or more medical imaging analysis tasks based on the image embeddings extracted from the one or more input medical images using a trained vision-language model; and outputting results of the one or more medical imaging analysis tasks, wherein the trained vision-language model is trained by: receiving one or more training medical images and a text-based report associated with the one or more training medical images, extracting image embeddings from the one or more training medical images, generating one or more instructions based on the text-based report using a language model, and training the vision-language model to perform the one or more medical imaging analysis tasks based on the image embeddings extracted from the one or more training medical images and the one or more generated instructions.

Illustrative embodiment 13. The non-transitory computer-readable storage medium of illustrative embodiment 12, wherein generating one or more instructions based on the text-based report using a language model comprises: generating the one or more instructions further based on a plurality of predefined templates, the plurality of predefined templates comprising different initial instructions for extracting information from the text-based report and generating the one or more instructions.

Illustrative embodiment 14. The non-transitory computer-readable storage medium of any one of illustrative embodiments 12-13, wherein generating one or more instructions based on the text-based report using a language model comprises: generating the one or more instructions for associating textual anatomical descriptors with image findings of the one or more training medical images.

Illustrative embodiment 15. The non-transitory computer-readable storage medium of illustrative embodiment 12-14, wherein the image findings comprise quantitative image findings of the one or more input medical images.

Illustrative embodiment 16. The non-transitory computer-readable storage medium of any one of illustrative embodiments 12-15, wherein: generating one or more instructions based on the text-based report using a first language model comprises: generating instruction embeddings representing the one or more instructions; and training the vision-language model to perform the one or more medical imaging analysis tasks based on the image embeddings extracted from the one or more training medical images and the one or more generated instructions comprises: combining the image embeddings extracted from the one or more training medical images and the instruction embeddings, and generating results of the one or more medical imaging analysis tasks based on the combined image embeddings and instruction embeddings.

Illustrative embodiment 17. A computer-implemented method comprising: receiving one or more training medical images and a text-based report associated with the one or more training medical images; extracting image embeddings from the one or more training medical images; generating one or more instructions based on the text-based report using a language model; and training a vision-language model to perform one or more medical imaging analysis tasks based on the image embeddings extracted from the one or more training medical images and the one or more generated instructions.

Illustrative embodiment 18. The computer-implemented method of illustrative embodiment 17, wherein generating one or more instructions based on the text-based report using a language model comprises: generating the one or more instructions further based on a plurality of predefined templates, the plurality of predefined templates comprising different initial instructions for extracting information from the text-based report and generating the one or more instructions.

Illustrative embodiment 19. The computer-implemented method of any one of illustrative embodiments 17-18, wherein generating one or more instructions based on the text-based report using a language model comprises: generating the one or more instructions for associating anatomical features depicted in the one or more training medical images with textual anatomical descriptors.

Illustrative embodiment 20. The computer-implemented method of any one of illustrative embodiments 17-19, wherein generating one or more instructions based on the text-based report using a language model comprises: generating the one or more instructions for associating anatomical features depicted in the one or more training medical images with each other.

1. A computer-implemented method comprising:
receiving one or more input medical images;
extracting image embeddings from the one or more input medical images;
performing one or more medical imaging analysis tasks based on the image embeddings extracted from the one or more input medical images using a trained vision-language model; and
outputting results of the one or more medical imaging analysis tasks,
wherein the trained vision-language model is trained by:
receiving one or more training medical images and a text-based report associated with the one or more training medical images,
extracting image embeddings from the one or more training medical images,
generating one or more instructions based on the text-based report using a language model, and
training the vision-language model to perform the one or more medical imaging analysis tasks based on the image embeddings extracted from the one or more training medical images and the one or more generated instructions.

2. The computer-implemented method of claim 1, wherein generating one or more instructions based on the text-based report using a language model comprises:
generating the one or more instructions further based on a plurality of predefined templates, the plurality of predefined templates comprising different initial instructions for extracting information from the text-based report and generating the one or more instructions.

3. The computer-implemented method of claim 1, wherein generating one or more instructions based on the text-based report using a language model comprises:
generating the one or more instructions for associating anatomical features depicted in the one or more training medical images with textual anatomical descriptors.

4. The computer-implemented method of claim 1, wherein generating one or more instructions based on the text-based report using a language model comprises:
generating the one or more instructions for associating anatomical features depicted in the one or more training medical images with each other.

5. The computer-implemented method of claim 1, wherein generating one or more instructions based on the text-based report using a language model comprises:
generating the one or more instructions for associating textual anatomical descriptors with image findings of the one or more training medical images.

6. The computer-implemented method of claim 5, wherein the image findings comprise quantitative image findings of the one or more input medical images.

7. The computer-implemented method of claim 1, wherein:
generating one or more instructions based on the text-based report using a language model comprises:
generating instruction embeddings representing the one or more instructions; and
training the vision-language model to perform the one or more medical imaging analysis tasks based on the image embeddings extracted from the one or more training medical images and the one or more generated instructions comprises:
combining the image embeddings extracted from the one or more training medical images and the instruction embeddings, and
generating results of the one or more medical imaging analysis tasks based on the combined image embeddings and instruction embeddings.

8. An apparatus comprising:
means for receiving one or more input medical images;
means for extracting image embeddings from the one or more input medical images;
means for performing one or more medical imaging analysis tasks based on the image embeddings extracted from the one or more input medical images using a trained vision-language model; and
means for outputting results of the one or more medical imaging analysis tasks,
wherein the trained vision-language model is trained by:
receiving one or more training medical images and a text-based report associated with the one or more training medical images,
extracting image embeddings from the one or more training medical images,
generating one or more instructions based on the text-based report using a language model, and
training the vision-language model to perform the one or more medical imaging analysis tasks based on the image embeddings extracted from the one or more training medical images and the one or more generated instructions.

9. The apparatus of claim 8, wherein generating one or more instructions based on the text-based report using a language model comprises:
generating the one or more instructions further based on a plurality of predefined templates, the plurality of predefined templates comprising different initial instructions for extracting information from the text-based report and generating the one or more instructions.

10. The apparatus of claim 8, wherein generating one or more instructions based on the text-based report using a language model comprises:
generating the one or more instructions for associating anatomical features depicted in the one or more input medical images with textual anatomical descriptors.

11. The apparatus of claim 8, wherein generating one or more instructions based on the text-based report using a language model comprises:
generating the one or more instructions for associating anatomical features depicted in the one or more input medical images with each other.

12. A non-transitory computer-readable storage medium comprising instructions which, when executed by a computer, cause the computer to carry out operations comprising:
receiving one or more input medical images;
extracting image embeddings from the one or more input medical images;
performing one or more medical imaging analysis tasks based on the image embeddings extracted from the one or more input medical images using a trained vision-language model; and
outputting results of the one or more medical imaging analysis tasks,
wherein the trained vision-language model is trained by:
receiving one or more training medical images and a text-based report associated with the one or more training medical images,
extracting image embeddings from the one or more training medical images,
generating one or more instructions based on the text-based report using a language model, and
training the vision-language model to perform the one or more medical imaging analysis tasks based on the image embeddings extracted from the one or more training medical images and the one or more generated instructions.

13. The non-transitory computer-readable storage medium of claim 12, wherein generating one or more instructions based on the text-based report using a language model comprises:
generating the one or more instructions further based on a plurality of predefined templates, the plurality of predefined templates comprising different initial instructions for extracting information from the text-based report and generating the one or more instructions.

14. The non-transitory computer-readable storage medium of claim 12, wherein generating one or more instructions based on the text-based report using a language model comprises:
generating the one or more instructions for associating textual anatomical descriptors with image findings of the one or more training medical images.

15. The non-transitory computer-readable storage medium of claim 14, wherein the image findings comprise quantitative image findings of the one or more input medical images.

16. The non-transitory computer-readable storage medium of claim 12, wherein:
generating one or more instructions based on the text-based report using a language model comprises:
generating instruction embeddings representing the one or more instructions; and
training the vision-language model to perform the one or more medical imaging analysis tasks based on the image embeddings extracted from the one or more training medical images and the one or more generated instructions comprises:
combining the image embeddings extracted from the one or more training medical images and the instruction embeddings, and
generating results of the one or more medical imaging analysis tasks based on the combined image embeddings and instruction embeddings.

17. A computer-implemented method comprising:
receiving one or more training medical images and a text-based report associated with the one or more training medical images;
extracting image embeddings from the one or more training medical images;
generating one or more instructions based on the text-based report using a language model; and
training a vision-language model to perform one or more medical imaging analysis tasks based on the image embeddings extracted from the one or more training medical images and the one or more generated instructions.

18. The computer-implemented method of claim 17, wherein generating one or more instructions based on the text-based report using a language model comprises:
generating the one or more instructions further based on a plurality of predefined templates, the plurality of predefined templates comprising different initial instructions for extracting information from the text-based report and generating the one or more instructions.

19. The computer-implemented method of claim 17, wherein generating one or more instructions based on the text-based report using a language model comprises:
generating the one or more instructions for associating anatomical features depicted in the one or more training medical images with textual anatomical descriptors.

20. The computer-implemented method of claim 17, wherein generating one or more instructions based on the text-based report using a language model comprises:
generating the one or more instructions for associating anatomical features depicted in the one or more training medical images with each other.
